(19) United States
(12) Patent Application Publication
(10) Pub. No.: US 2024/0379249 A1
(43) Pub. Date: Nov. 14, 2024

(54) METHOD AND SYSTEM FOR COLLECTING, RECORDING, AND TRANSMITTING CLINICAL INFORMATION WITH USEFULNESS-DRIVEN PROMPTS

(71) Applicant: SimulConsult, Inc., Chestnut Hill, MA (US)

(72) Inventor: Michael M. SEGAL, Chestnut Hill, MA (US)

(21) Appl. No.: 18/696,720

(22) PCT Filed: Sep. 29, 2022
(86) PCT No.: PCT/US2022/045276

§ 371 Date: Mar. 28, 2024

Related U.S. Application Data
(60) Provisional application No. 63/249,675, filed on Sep. 29, 2021.

(51) Int. Cl. G16H 80/00; G16H 10/40; G16H 20/00; G16H 40/20; G16H 50/20

(52) U.S. Cl. CPC G16H 80/00; G16H 10/40; G16H 20/00; G16H 40/20; G16H 50/20

(57) ABSTRACT

The present invention relates to a system and methods for improving characterization of the patient's symptoms. signs and test results and sharing it among the care team in a way that maximizes clinician productivity and improves patient outcomes.

### BACKGROUND

The practice of medicine is difficult and time consuming as there are thousands of diseases, some newly discovered, for practitioners to distinguish. Diseases have “findings,” such as symptoms, signs, test results, and historical information, that also occur in other diseases, so diagnosis is a pattern-matching process. Clinicians cannot know what findings might be useful in diagnosing a particular patient if they do not remember the relevant disease or have not even learned about it.

Clinicians have used decision trees to guide diagnosis in some common situations, such as workups for particular common problems such as seizures that can be part of unusual conditions. Clinicians have also used static lists for findings that are important for particular medical specialties. But decision trees are suboptimal because they force the clinician to proceed in a fixed sequence to collect findings. Static lists are suboptimal because they don't guide clinicians as to which of the many findings are useful to collect, and collecting all is time consuming or expensive. These existing approaches seldom take into account the many diseases that can occur at different ages, and these approaches fail to prioritize information that is needed based on information that has been entered.

What clinicians need is a clinical computing system that can handle these common situations in a way that allows for rapid clinician workflow and reason alongside the clinician dynamically.

Today, when a diagnostic referral is appropriate, little information is passed along in the referral, making it difficult to triage the appointment for urgency, choose the appropriate specialist to be seen, and recommend useful tests to be done by the referring clinician before that appointment. A system that guides the clinician to include a robust set of pertinent findings in the referral would be an advance over current practice. A system that does so efficiently, without the bloat of non-pertinent information as is characteristic of many tools in electronic health record (EHR) patient charts, and is interoperable among different EHRs would be a further advance over current practice.

### SUMMARY OF THE INVENTION

The present invention features systems and methods for improving characterization of the patient's symptoms, signs, and test results and being able to share that information among the care team in a way that maximizes clinician productivity and improves patient outcomes.

In one aspect, the invention provides a method including the steps of: (a) providing a physical computing device having stored therein a plurality of candidate medical conditions and a plurality of “workup lists” of findings wherein each of the findings in a particular list is representative of clinical information about particular common medical condition or the wider set of medical conditions seen by that specialty and wherein for both types of workup lists, each finding in the list of findings can be tagged with a metric of the usefulness of the finding, (b) specifying in the physical computing device a finding in a workup list of findings as being present or absent in a patient, wherein the physical computing device automatically re- tags the findings in the list of findings with a metric of usefulness of the finding in disambiguating among the plurality of medical conditions resulting from the list of findings specified by a user as being present or absent in the patient.

In a related aspect, the invention provides a method including the steps of:
(a) providing a physical computing device having stored therein a plurality of candidate medical conditions and lists of findings for a plurality of problem or medical specialty workups, wherein each of the findings is representative of clinical information about the medical conditions, and wherein the findings in the lists of findings are tagged with a metric of the usefulness of the finding in disambiguating among the plurality of medical conditions;
(b) specifying in the physical computing device a first finding as being present or absent in a patient in a first list of findings of a workup of the plurality of problem or medical specialty workups, wherein the physical computing device then automatically re-tags the findings in the first list of findings as a function of the usefulness of each finding in disambiguating among the plurality of medical conditions computed based on the specifying of the first finding; and
(c) specifying in the physical computing device another finding either in the first list of findings or in a second list of findings of another problem or medical specialty workup as being present or absent in the patient, and recomputing the usefulness of unspecified findings in the first or second list.

The method may further include the steps of (d) outputting a report of the findings specified in (b) and (c) indicating whether the finding is absent, or present organized by a computed rank of pertinence; and optionally (e) transmitting the report to a second user. Pertinence indicates the importance of the finding in the calculation of the differential diagnosis, and therefore, indicating to the clinician the findings where it is most important to be confident in their presence or absence. In some embodiments, a specified finding is tagged as a pertinent positive or a pertinent negative

In some embodiments, the user is a primary care clinician. In some embodiments, the second user is a specialist in the first medical specialty. The method may further include generating in the physical computing device a list of medical conditions ranked by likelihood, called a “differential diagnosis”. In some embodiments, the report comprises a character string that allows re-creation of the list of medical conditions ranked by likelihood. In some embodiments, in step (c), the another finding is in the first list of findings. In some embodiments, the method further includes specifying in the physical computing device the age and/or sex of a subject. In some embodiments, the method further includes specifying an onset of the first or the another finding, when present, since timing of onset is a key characteristic of many medical conditions. In some embodiments, the method further includes the step of ordering via the physical computing device a medical test to determine the presence or absence of an unspecified finding in the first or second list of findings. In some embodiments, the report includes structured coding of the first and another findings to standardized coding lists. In some embodiments, the report is in the Subjective, Objective, Assessment, and Plan (SOAP) note format. In some embodiments, the method further includes the step of computing the pertinence of findings specified in (b) and (c) in distinguishing among the plurality of medical conditions. In some embodiments, the method further includes the step of displaying a list of the medical tests tagged with a metric of usefulness in disambiguating among the plurality of medical conditions or a list of a subset of medical tests selected by the user. In some embodiments, the medical specialty is genetics, metabolism, mitochondrial, neurology, rheumatology, allergy, immunology, anesthesiology, dermatology, diagnostic radiology, emergency medicine, family medicine, internal medicine, nuclear medicine, obstetrics, gynecology, ophthalmology, pathology, pediatrics, physical medicine and rehabilitation, preventive medicine, psychiatry, radiation oncology, surgery, or urology. In some embodiments, the report includes plain text that can be copied and pasted.

In some embodiments, in step (c) the another finding is in the second list. In some embodiments, the physical computing device highlights the name of the another workup to the user based on the usefulness of the findings in its list of findings to further characterize the patient.

In another aspect, the invention provide a physical computing device having stored therein a plurality of candidate medical conditions and lists of findings for a plurality of problem or medical specialty workups, wherein each of the findings is representative of clinical information about the medical conditions and being programmed with executable instructions for (a) allowing a user to specify a first finding in a first list of findings for a workup of the plurality of problem or medical specialty workups as being present or absent in a patient; (b) calculating the usefulness of unspecified findings in the first list or a second list of findings of another problem or medical specialty workup based on the first finding; and (c) allowing the user to specify in the physical computing device another finding either in the first list of findings or in the second list of findings, and recomputing the usefulness of unspecified findings in the first or second list.

In some embodiments, in (a) the workup is a medical specialty workup, and in (c) the another finding is in the second list. In some embodiments, the device is programmed to output a report of the findings from the first list indicating whether the finding is present, absent, or unknown, organized by pertinence rank. In some embodiments, the device is further programmed to compute the pertinence of findings specified in (a) and (c) in distinguishing among the plurality of medical conditions. In some embodiments, the device is further programmed to display a list of medical tests tagged with a metric of usefulness in disambiguating among the plurality of medical conditions or a list of medical tests selected by the user. In some embodiments, the device is further programmed to determine the usefulness of an unspecified finding in the second list and highlighting the name of the another workup to the user based on the usefulness of the findings in its list. In some embodiments, the device is further programmed to perform at least one of the following steps:

calculate and display a differential diagnosis;

create a report comprising computable information on a status of findings in the first and/or second lists;

create a report comprising a status and/or usefulness of the first finding;

create a report comprising a status and/or usefulness of all findings specified in (a) and (c);

create a report that is in SOAP note format;

create a report comprising computable information on a status of the findings specified in (a) and/or (c);

share a report with a second user; and/or

transfer computable information on a status of the findings specified in (a) and (c) from a report into the device.

In another aspect, the invention provides a non-transient computer readable medium having stored therein a plurality of candidate medical conditions and lists of findings for a plurality of problem or medical specialty workups, wherein each of the findings is representative of clinical information about the medical conditions and executable instructions to direct a physical computing device to implement a method comprising (a) allowing a user to specify a first finding in a first list of findings for a workup of the plurality of problem or medical specialty workups as being present or absent in a patient; (b) calculating the usefulness of unspecified findings in the first list or a second list of findings of another problem or medical specialty workup based on the first finding; and (c) allowing the user to specify in the physical computing device another finding either in the first list of findings or in the second list of findings, and recomputing the usefulness of unspecified findings in the first or second list.

In some embodiments, in (a) the workup is a medical specialty workup, and in (c) the another finding is in the second list. In some embodiments, the instructions further direct the device to output a report of the findings in the first list indicating whether the finding is present, absent, or unknown, organized by pertinence rank. In some embodiments, the instructions further direct the device to compute the pertinence of the findings specified in (a) and (c) in distinguishing among the plurality of medical conditions. In some embodiments, the instructions further direct the device to display a list of medical tests tagged with a metric of usefulness in disambiguating among the plurality of medical conditions or a list of medical tests selected by the user. In some embodiments, the instructions further direct the device to determine the usefulness of an unspecified finding in the second list and highlight the name of the another workup to the user based on the usefulness of the findings in its list. In some embodiments, the instructions further direct the device to perform at least one of the following steps:

calculate and display a differential diagnosis;

create a report comprising computable information on a status of findings in the first and/or second lists;

create a report comprising a status and/or usefulness of all findings specified in (a) and (c);

create a report that is in SOAP note format;

create a report comprising computable information on a status of the findings specified in (a) and/or (c);

share a report with a second user; and/or

transfer computable information on a status of the findings specified in (a) and (c) from a report into the device.

In another aspect, the invention provides a method of facilitating medical care including

(a) providing a physical computing device as described herein; and

(b) specifying in the physical computing device a first finding as being present or absent in a patient in a first list of findings for a workup;

(c) calculating the usefulness of unspecified findings in the first list or a second list of findings of another problem or medical specialty workup based on the first finding; and

(d) specifying in the physical computing device another finding either in the first list of findings or in the second list of findings, and recomputing the usefulness of unspecified findings in the first or second list.

In some embodiments, the physical computing device automatically re-tags the findings in the first list of findings as a function of the usefulness of each finding in disambiguating among the plurality of medical conditions computed based on the specifying of the first finding. In some embodiments, the method further includes outputting a report of the findings from the first list indicating whether the finding is present, absent, or unknown, organized by pertinence rank. In some embodiments, the method further includes transmitting the report to a second user. In some embodiments, the method further includes generating in the physical computing device a list of medical conditions ranked by likelihood. In some embodiments, the report includes a character string that allows re-creation of the list of medical conditions ranked by likelihood. In some embodiments, in step (d) the another finding is in the first list of findings. In some embodiments, the method further includes specifying in the physical computing device the age and/or sex of a subject. In some embodiments, the method further includes specifying an onset of the first or another finding, when present. In some embodiments, the method further includes the step of ordering via the physical computing device a medical test to determine the presence or absence of an unspecified finding in the first or second list of findings. In some embodiments, the report includes structured coding of the findings specified in (a) and (d) to standardized coding lists. In some embodiments, the report is in the Subjective, Objective, Assessment, and Plan (SOAP) note format. In some embodiments, the method further includes the step of computing the pertinence of the findings specified in (a) and (c) in distinguishing among the plurality of medical conditions. In some embodiments, the method further includes the step of displaying a list of the medical tests tagged with a metric of usefulness in disambiguating among the plurality of medical conditions or a list of medical tests selected by the user. In some embodiments, the report includes a character string that allows re-creation of the list of medical conditions ranked by likelihood. In some embodiments, the report comprises plain text that can be copied and pasted. In some embodiments, the another finding is in the second list. In some embodiments, the physical computing device highlights the name of the another workup to the user based on the usefulness of the findings in its list of findings to further characterize the patient. In some embodiments,

In another aspect, the invention provides a non-transitory computer readable medium having stored therein (a) a plurality of candidate medical conditions; (b) lists of findings for a plurality of medical specialties, wherein each of the findings is representative of clinical information about the medical conditions, and wherein the findings in the lists of findings are tagged with a metric of the usefulness of the finding in disambiguating among the plurality of medical conditions; and (c) instructions for causing one or more processors to execute steps of a method described herein.

In another aspect, the invention provides a system including a physical computing device having one or more processors, a network communication interface, and one or more computer readable memories having stored therein a plurality of candidate medical conditions; lists of findings for a plurality of medical problems and specialties, wherein each of the findings is representative of clinical information about the medical problems or specialty, and wherein the findings in the lists of findings are tagged with a metric of the usefulness of the finding in disambiguating among the plurality of diseases; and instructions that when executed by the one or more processors cause the system to carry out the steps of a method described herein.

### BRIEF DESCRIPTION OF THE DRAWINGS

FIG. 1 shows stages in conventional medical thinking.

FIG. 2 shows the average clinician time spent in minutes per patient per incident, including all stages of medical thinking and documentation, with and without the present invention.

FIG. 3 shows the impact on quality on more complex cases with and without the present invention.

FIG. 4 shows the selection of a first finding in a physical computing device having stored therein a plurality of candidate medical conditions and lists of findings for a plurality of workups (problems or medical specialties), e.g., entering the first finding of kyphosis in the Genetics specialty workup.

FIG. 5 shows changes in usefulness (shown as shading in the name of the finding) after adding a first finding, kyphosis. The usefulness metric for various findings such as macrocephaly has increased.

FIG. 6 shows changes in usefulness after specifying the presence of macrocephaly in addition to the kyphosis. The usefulness metric for corneal clouding has increased.

FIG. 7 shows changes in usefulness after adding a third finding, corneal clouding.

FIG. 8 shows changes in usefulness after adding one pertinent negative finding (lack of short stature).

FIG. 9 shows an automated report of the findings, with a metric of pertinence assigned to each finding.

FIG. 10 shows an example of exchange of automated, plain text communications about a referral from a primary care clinician to a specialist (left) and a response to the primary care clinician requesting a further test (right), both using automated reports from the software.

FIG. 11 shows inputting a patient text string to reload the patient (top) and examining the resulting differential diagnosis of likely diseases, with shading indicating relative probability.

FIG. 12 shows prompts with shading corresponding to the usefulness metric that help the clinician decide on further tests that draw upon the full list of all the many thousands of findings, far beyond the findings in the plurality of problem workups or medical specialty workups.

FIG. 13 shows the list of findings for the Psychosis problem workup, spanning many medical specialties and using both clinical and lab findings.

### DEFINITIONS

As used herein, the term “finding” refers physical and psychological occurrences surveyed by a clinician and may include symptoms, impairments, physical attributes, mental attributes, test results, historical information, etc.

As used herein, the term “differential diagnosis” refers to the ranked list of candidate medical conditions that a patient may have, based on the findings collected, a list that evolves as more information is collected.

As used herein, the term “usefulness” refers to a prospective metric of the degree of findings to distinguish among known diseases, e.g., as a function of the likelihood of being relevant, cost-effective and focusing more on diseases that are treatable, giving advice as to what information is most important to collect.

As used herein, the term “pertinence” refers to a retrospective metric of the relevance that a finding that was specified for the patient had in distinguishing among medical conditions, showing what findings were most heavily relied on in the analysis and thus identifying the findings for which it is most important to be sure of the information.

As used herein, the term “clinician” means a Medical Doctor (MD), Doctor of Osteopathic Medicine (DO), nurse practitioner, physician's assistant, licensed practical nurse, registered nurse, and any other licensed medical professional.

As used herein, the term “primary care clinician” refers to the clinician primarily responsible for a patient's care, and includes primary care physicians, nurse practitioners, physician's assistants, etc.

As used herein, the term “workup” (often abbreviated w/u) refers to a set of findings used to approach certain common medical problems or less common situations in various medical specialties.

As used herein, the term “medical specialties” refers to branches of medical practice that are focused on a defined group of patients, diseases, skills, or philosophy.

As used herein, the term “problem” refers to medical problems that are common enough to have defined approaches but may span multiple specialties and many diseases. Some may be handled entirely by primary care, while others are rarer or more difficult to diagnosis, and therefore typically diagnosed by specialists; the clinician's diagnostic task is to disambiguate among these possibilities.

### DETAILED DESCRIPTION

The present invention provides methods and related systems that may be used to aid clinicians in characterizing patient findings, making progress towards a diagnosis and sharing the findings that were collected with other clinicians, e.g., specialists, in a way that is computable.

The present invention employs centralized and computable medical knowledge. The present invention employs databases of medical conditions and findings useful in making a diagnosis. The invention determines a likelihood of each medical condition based on patient findings and reasons alongside the clinician at each stage of medical decision making, while allowing the clinician to be the final authority in making a diagnosis.

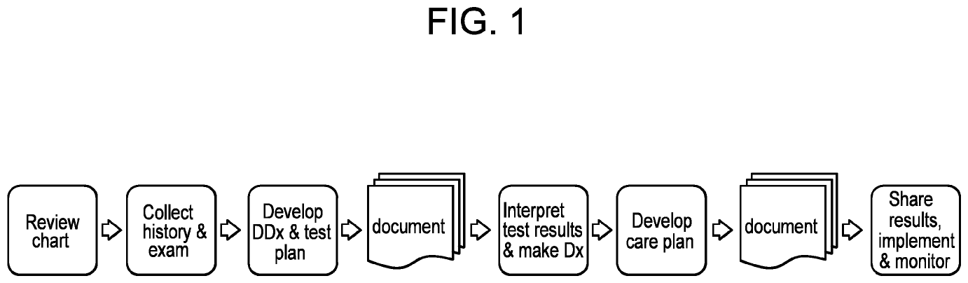

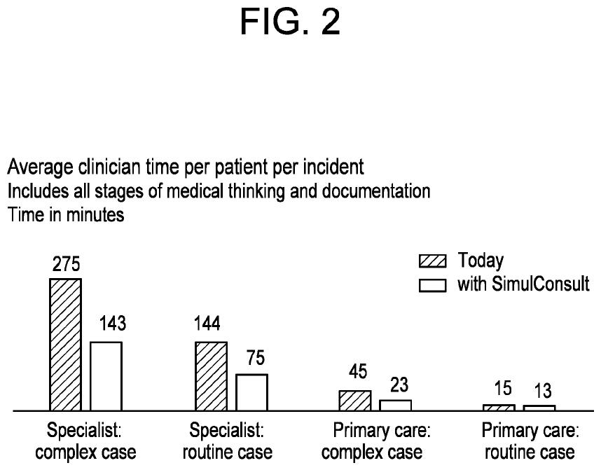

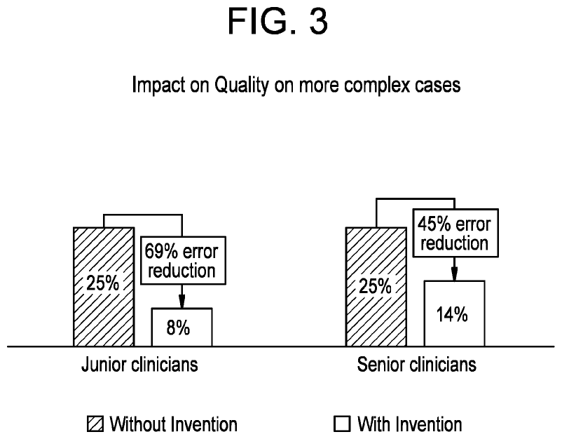

The number of known medical conditions increases 5-10% each year, leaving clinicians to memorize or research an ever growing number of medical conditions when diagnosing patients. This problem is particularly apparent with primary care clinicians, who are generalists, but need to be able to refer patients to specialists for care in appropriate circumstances. FIG. 1 shows stages in conventional medical thinking. The present invention significantly speeds clinician time spent per patient per incident, as shown in FIG. 2, as well as increases the quality of clinician diagnosis, as shown in FIG. 3.

The present invention streamlines the process for diagnosis by employing problem or medical specialty workups, each of which includes a selected list of potential findings. The clinician can then rapidly select for the presence or absence of findings within the selected list for a particular workup or in multiple workups, if desired. This is much faster than wider approaches that use orders of magnitudes more findings, many of which will be unfamiliar to primary care clinicians.

In addition, the present invention solves a problem of communication between medical providers. In particular, the invention allows a non-specialist to specify useful findings, with the aid of a usefulness metric, and transmit this information quickly and concisely to a specialist. The detailed information in a concise format that is computable allows the specialist to review the findings by looking at the report or importing the computable report into the software, and make quick decisions on additional testing, which may be requested via the original, non-specialist, and the urgency of a consultation. The invention can also determine a differential diagnosis, allowing the clinician to consider whether a diagnostic referral is needed. This information is computable in that it can be transmitted between clinicians using a text string (or other code such as a barcode or QR code) that may be included in the report to allow the re-creation of the differential diagnosis for use by a clinician receiving the referral or by the original clinician re-opening the same case in the future. Furthermore, the report generation can be automatic during the input of various findings or ordering of additional tests. At the end of analysis, the report may be sent quickly and easily to a specialist for consideration. All of these features focus the information transmitted back and forth on the more pertinent findings already determined or the most useful findings yet to be determined.

### The System

In some embodiments, the present invention includes a physical computing device having stored therein a plurality of candidate medical conditions and lists of findings for a plurality of problem or medical specialty workups. Each list of findings for a common medical problem or medical specialty includes a plurality of findings, e.g., selected to be those that a primary care clinician should be able to assess. The specialty workup lists include findings most used on referrals to specialty clinicians, and the common clinical problem workup lists include findings most used to make progress towards diagnosis of a common clinical problem. In some embodiments, the findings in the lists of findings get tagged with a metric of the usefulness of the finding in disambiguating among the plurality of medical conditions, which changes with each new finding added. An exemplary platform for use with the invention is SimulConsult (Chestnut Hill, MA), aspects of which are described in U.S. Pat. No. 6,754,655, 9,524,373, and WO 2021/159054, each of which is incorporated herein by reference in its entirety.

In some embodiments, common medical problems include, but are not limited to abdominal pain, autism, child abuse, coma, developmental delay, failure to thrive, headache, hypoglycemia, hypotonia, macrocephaly, microcephaly, psychosis, seizure, stature short, and/or urinary incontinence.

In some embodiments, medical specialties include, but are not limited to, genetics, metabolism, mitochondrial, neurology, rheumatology, allergy, immunology, anesthesiology, dermatology, diagnostic radiology, emergency medicine, family medicine, internal medicine, nuclear medicine, obstetrics, gynecology, ophthalmology, pathology, pediatrics, physical medicine and rehabilitation, preventive medicine, psychiatry, radiation oncology, surgery, and/or urology.

In some embodiments, physical computing device has stored therein a list of medical tests of which the clinician may view or order.

The invention provides systems to carry out the methods of the invention and non-transitory computer readable media having stored therein instructions and data for carrying out the methods of the invention.

Systems include a physical computing device with one or more processors, a network communication interface, and one or more computer readable memories to store data and instructions for carrying out the methods of the invention.

The physical computing device may be implemented in any suitable manner. For example, it may reside on a single server or computer or be distributed across multiple computers or servers, e.g., in a cloud architecture. The invention may be accessed by a standard desktop or terminal by a dedicated program or webpage. The invention may also be accessed via a mobile application.

The network communication interface may allow communication between the physical computing device and the user or between multiple users, e.g., primary care clinician and specialist. The interface may also allow communication between network components of the physical computing device. The network communication interface may also be implemented as several different components, e.g., one for communication with the user and one for communication with sources of electronic patient information. Any standard network communication protocol may be employed, e.g., Transmission Control Protocol (TCP), Internet Protocol (IP). Global System for Mobile Communications (GSM) based cellular network, Wi-Fi, Bluetooth, and Near Field Communication (NFC). Connections may also be wired, wireless, or a combination thereof.

Any suitable computer readable memory or non-transitory computer readable medium may be employed in the physical computing device. Such memories and media include magnetic disks, optical disks, organic memory, and any other volatile (e.g., Random Access Memory (RAM), flash, and EEPROM) or non-volatile (e.g., Read-Only Memory (ROM)) mass storage system readable by the one or more processors. The memory or medium includes standalone or cooperating or interconnected memories or media, which may be distributed among multiple interconnected computers or servers that may be local or remote. In one embodiment, the data are stored with one or more encryption and/or security methods. A system may also include any other components necessary for operation, e.g., displays, switches, and routers.

### Operation

The present invention transforms the ability of a clinician to characterize the patient quickly and easily in the situations most commonly experienced by primary care clinicians and decide what to do next, for example decide whether a) it's normal, so do nothing; it might be something, so do “watchful waiting;” b) we know what it is, so move on to treatment; or c) it's rare or unclear, so refer to the specialty that most matches the symptoms and signs. In addition, the present invention facilitates transfer of patient findings from one clinician to another, for example to and from a primary care clinician and a specialist.

The user can select a workup for a common problem or a workup for a medical specialty, which provides a reduced set of findings for consideration. In some embodiments, the number of findings for a particular workup is less than 100, e.g., less than 75 or less than 50. In contrast, the invention may include a total of over 10,000 findings, a number that can be overwhelming for a primary care clinician and include many findings that a primary care clinician would not be in a position to assess. The findings for each common problem or medical specialty will differ, but there may be some overlap in individual findings. The findings are preferably those easily ascertainable by a non-specialist. In some embodiments, the clinician inputs patient background such as height, weight, sex, and/or drug and alcohol history as findings. In some embodiments, the clinician may enter patient name, contact information, age, and/or prescriptions.

The clinician may characterize the patient quickly and easily by using the invention. The process starts by entering one or more findings in one of the workups. Following the entering of the first finding, the invention automatically re-tags the findings in the list of findings with a metric of the usefulness of the finding in disambiguating among the plurality of medical conditions deduced from the totality of the findings specified by the user as being present or absent in the patient. This points the clinician towards the findings for which it is useful to comment, without the clinician needing to know why each particular finding is useful. This process may be repeated for as many findings as desired, allowing the user to rapidly document useful information without the need to comment on the presence or absence of every finding in a list of findings.

The documentation of the status of findings may include not only findings present (“pertinent positives”) but also information about their onset and information about findings that are absent (“pertinent negatives”).

In some embodiments, the methods include displaying either a list of the most useful tests or only the tests that the user selects, e.g., by editing a plain text output. In some embodiments, the clinician may additionally order, e.g., via the physical computing device, a medical test to determine the presence or absence of a finding in the first list of findings.

In some embodiments, the methods include the step of automatically documenting the findings entered, with or without making visible the ranked list of candidate medical conditions (a “differential diagnosis”). The clinicians may re-open the case at any time using the output files or a computable character string that documents the findings specified, and review the case history and change the list of findings, for example adding a test result. In some embodiments, the method displays the information in the standard “SOAP” note format (Subjective, Objective, Assessment, and Plan) with or without the ranked list of candidate diseases.

The findings selected by the clinician may be output in a computable report file or a computable character string, allowing other members of the care team to start from where the first clinician left off, enabling a rapid electronic consult from the specialist to the primary care clinician, advising of additional useful tests and prioritizing patients as appropriate, with automatic documentation of the consult.

In some embodiments, the report includes a computable character string that allows re-creation by the initial or subsequent user of a list of medical conditions ranked by likelihood. In some embodiments, the report includes structured coding of the findings to standardized coding lists. In some embodiments the structured coding is contained in an optically readable form, such as a bar code or QR code. In some embodiments, a plain text report can be copied and put into any electronic health record email, patient note or referral request format, or the report may be printed.

In some embodiments, the report shows a graphical display of selected findings and their pertinence in disambiguating between a plurality of medical conditions. In some embodiments, the report separates pertinent positive findings from pertinent negative findings.

The present invention also provides novel pathways for clinician interaction. The invention provides for the findings and tests input by a first clinician to be shared with a second clinician. In some embodiments, the first clinician is a primary care clinician. In some embodiments, the second clinician is a medical specialist.

For example, a primary care clinician may assess a patient and decide to refer the patient to a specialist. The primary care clinician may input findings or test results from tests that have already been done on the patient while assessing the patient. The primary care clinician may then transfer the report of the findings and tests to the specialist, who may pick up assessment of the patient where the primary care clinician left off, reducing time in reassessment or analysis of the primary care clinician's thought process. The specialist may then input further findings and tests to be done on the patient, before transferring a computable report back to the primary care clinician, or another specialist. In this way, the present invention provides systems and methods for care teams to treat patients efficiently and effectively.

In some embodiments, the methods further include the step of transferring or transmitting the report from a first clinician to a second clinician. In some embodiments, the present methods further comprise the step of transferring or transmitting the report from the second clinician back to the first clinician.

The methods and systems may also include generating the pertinence of the findings that were specified by the user as being present or absent and outputting a list of such findings ranked by pertinence, a measure of the estimated contribution of that finding to driving the differential diagnosis (e.g., as described in U.S. Pat. No. 9,524,373, which is hereby incorporated by reference), making clear which findings are most strongly driving the most highly ranked diagnoses, and thus findings for which it is most important for the clinician user to be sure of their accuracy. The findings specified may also include genetic sequencing information associated with the patient, e.g., identification of one or more genetic variants, and for each of the one or more genetic variants, a measure of zygosity of such variants for the patient, wherein for each of said one or more genetic variants, a severity score is provided in the plurality of genetic findings or the computing device generates the severity score, and wherein estimated probabilities of the candidate diseases are generated using the severity scores for each of the one or more genetic variants (e.g., as described in U.S. Pat. No. 9,524,373).

### EXAMPLES

The following provides a non-limiting example of one implementation of the invention.

The invention as implemented in SimulConsult, a commercial product used by clinicians for assistance in making diagnoses (Segal M M. Appl Transl Genom. 2015; 6:26-27; Segal M M, et al. J Child Neurol. 2015 30:881-8; Segal M M, et al. J Child Neurol. 2014 29:487-492; Segal M M et al. Pediatric rheumatology online journal. 2016; 14:67) was used to evaluate a patient.

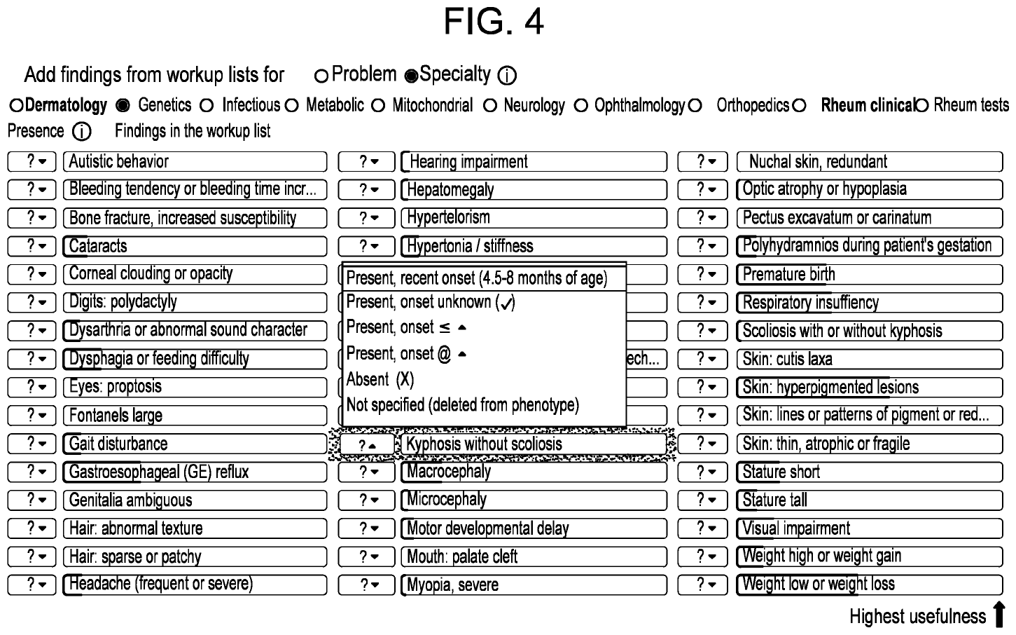

A first finding, kyphosis with recent onset, is entered using the Genetics medical specialty workup, for a patient who had been specified as being an 8-month-old boy, as shown in FIG. 4.

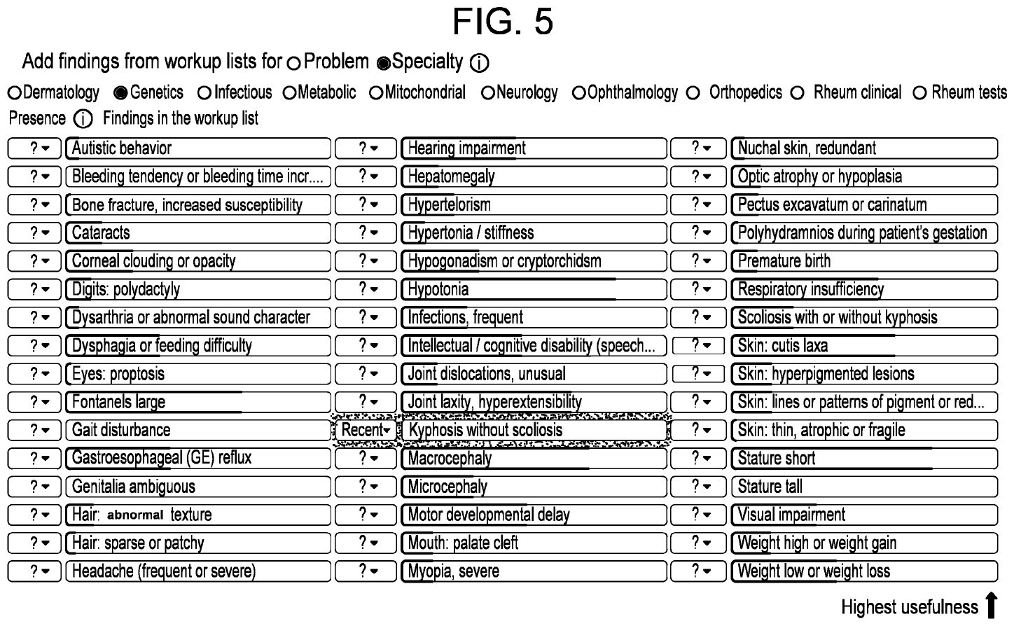

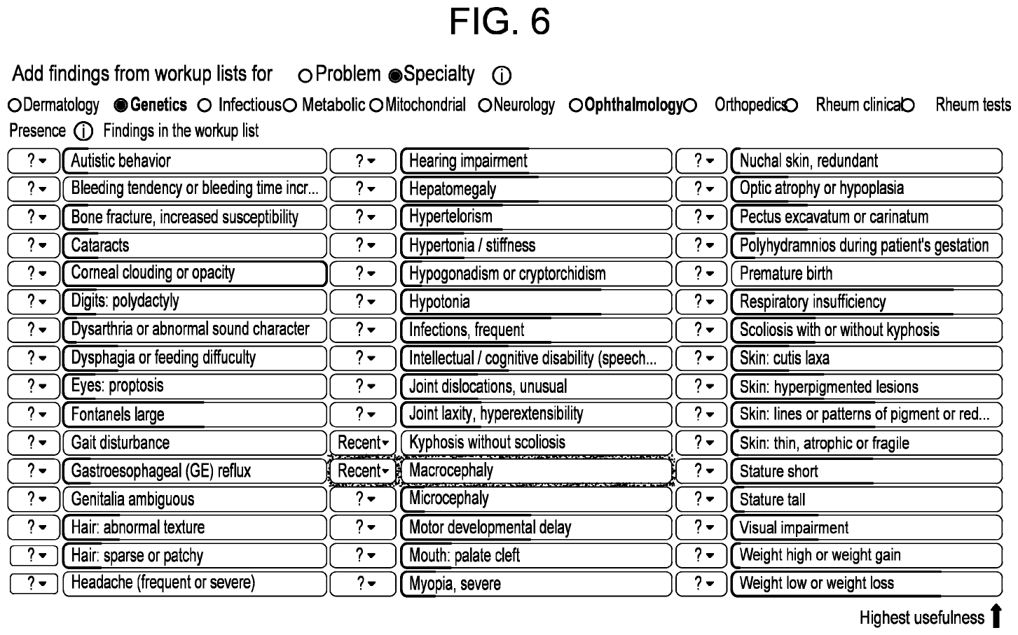

Once the first finding is entered, the usefulness of other findings is recomputed and displayed, as indicated by the shaded bars of different lengths on finding names as shown in FIG. 5. Among those findings with usefulness that is high and increased after specifying the first finding is macrocephaly (big head). As shown in FIG. 6, the process continues with the clinician accepting the advice that commenting on macrocephaly is useful, and specifying macrocephaly as present with recent onset. The usefulness of other findings is recomputed.

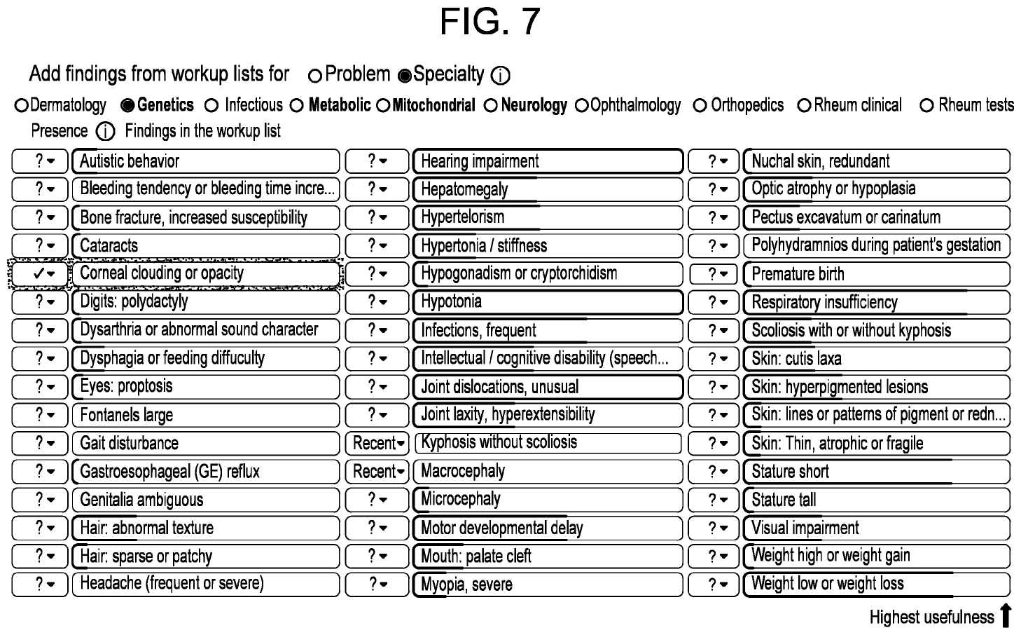

The clinician can continue to input information on as many additional findings as known or desired, using the usefulness as a guide to selecting useful findings and not needing to bother with documenting most of the other findings in the list. As shown in FIG. 7, the clinician has remained on the Genetics medical specialty workup but has specified that the ophthalmologic finding of corneal clouding is present, with unknown onset, and the usefulness of other findings is recomputed.

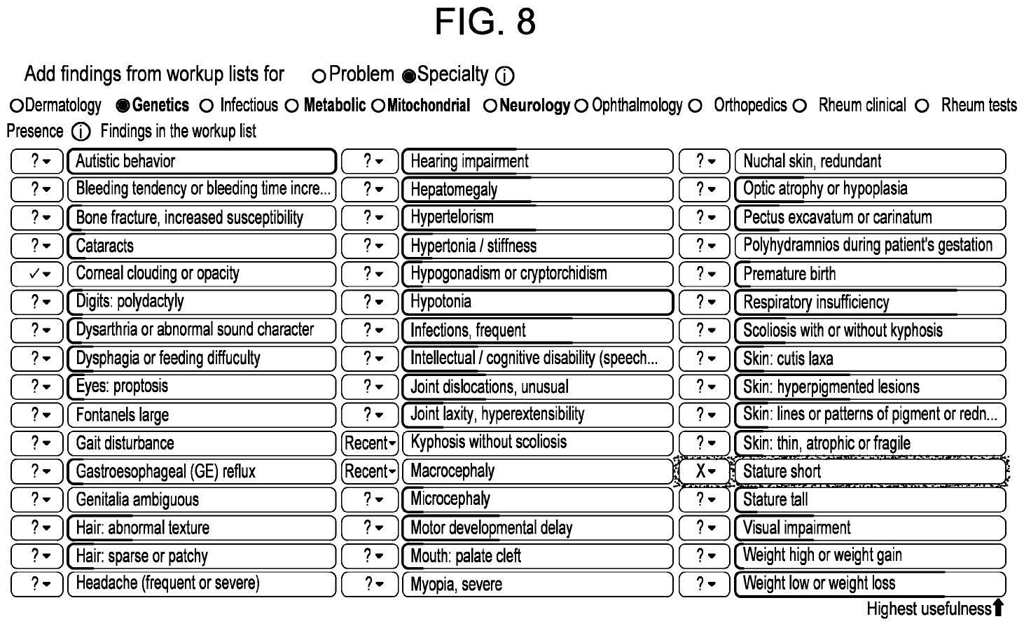

The short stature finding is now tagged as useful on FIG. 7, and on FIG. 8 it is specified as absent.

The clinician can also choose to shift to a different workup if, in light of findings added to date, uncommented findings on another workup findings list may be useful, e.g., as signaled in the invention by the name of the workup findings list being highlighted. As an example, in FIG. 6, the clinician could have noticed that the name of the Ophthalmology specialty workup list was also shown in bold, due to the corneal clouding finding also being on that workup list.

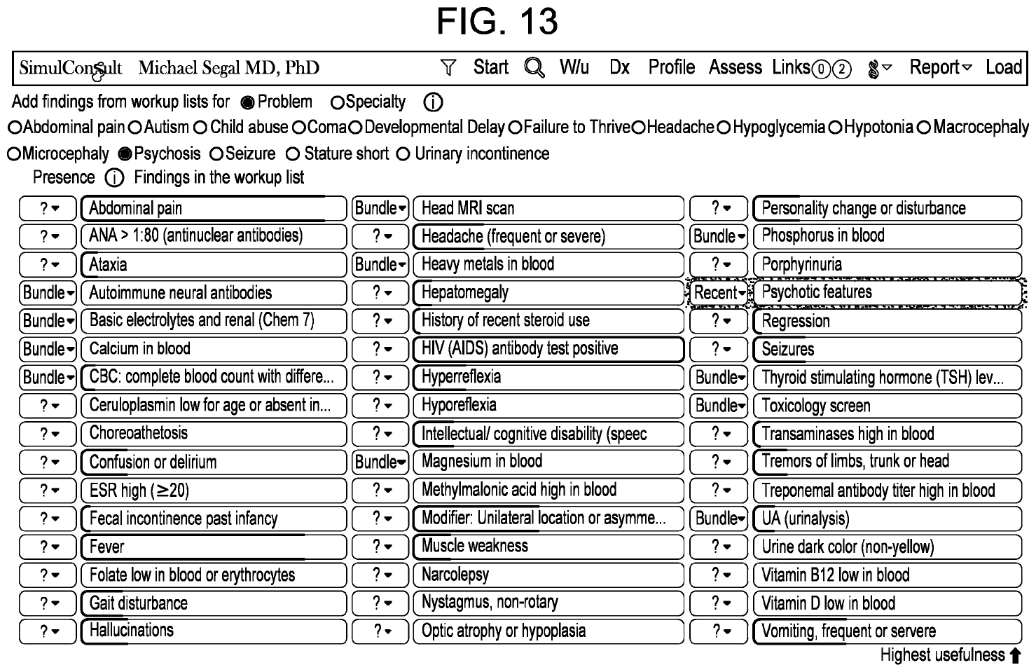

A workup list for a common clinical problem, psychosis, is shown in FIG. 13 and a similar process as illustrated in FIGS. 4-8 can be carried out. FIG. 13 also shows a list of many other workup lists for common clinical problems.

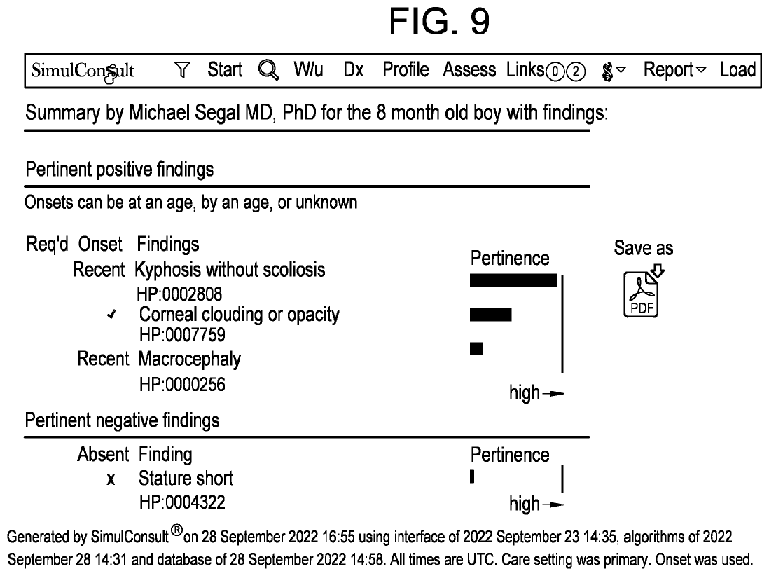

A primary care clinician could stop after this process, which requires less than a minute, and choose one of several options such as sending a report to a specialist, or choosing instead to order further tests, e.g., based on the usefulness of test result findings. One type of report, shown in FIG. 9, lists only the findings, avoiding sending a differential diagnosis that might prompt concerns in the recipient about being thus forced to test for each disease on such a list. The display provides the opportunity to download a PDF file that not only displays the information on this screen but contains a computable text string allowing the recipient to re-create the patient information in the software.

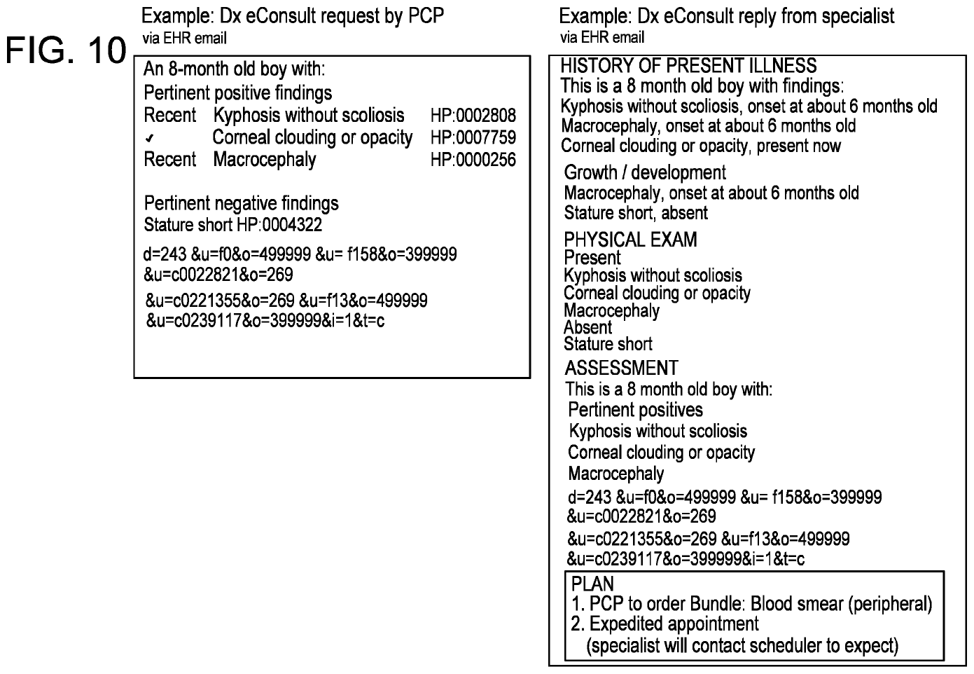

The communication may also be in plain text using electronic health record supported email. Such an exchange between the primary care clinician and the specialist would typically be the plain text automated output, shown in FIG. 10 (left). Standardized codes for the findings, such as Human Phenome Ontology (HIPO) codes may be provided as shown in FIG. 10. The report allows a specialist to quickly understand which findings are most pertinent. The specialist then considers the case and can respond, e.g., with a view on the urgency of the consultation and any further tests that would be helpful. An automated, customizable output documents the consult using the standard Subjective, Objective, Assessment, and Plan (SOAP) format.

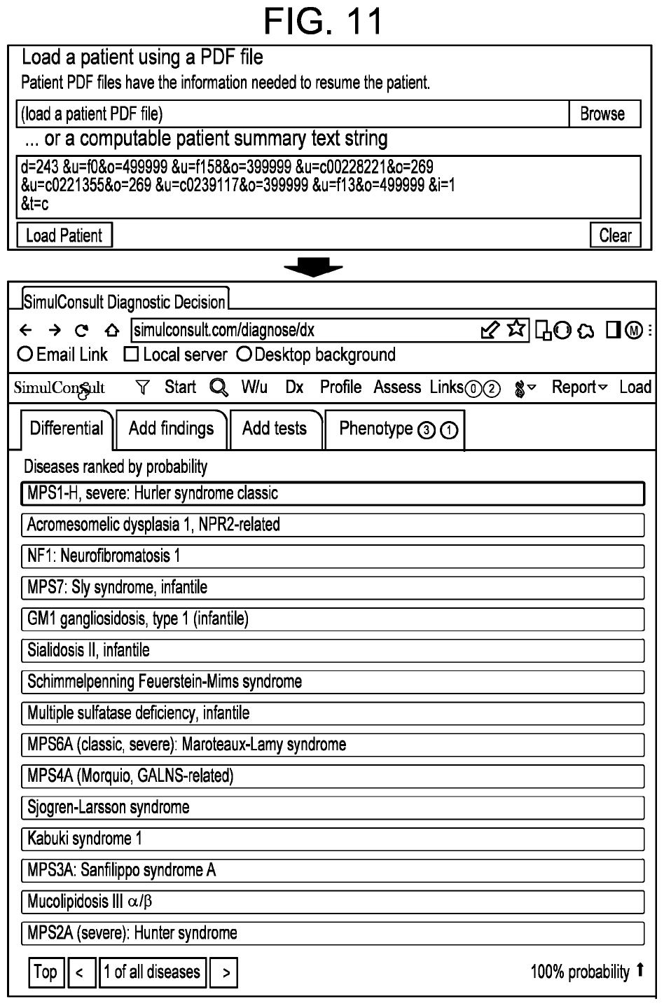

The text string and the bottom of FIG. 10 (left) can be used by either clinician as an input to re-create the case in the software, as shown in FIG. 11 (top).

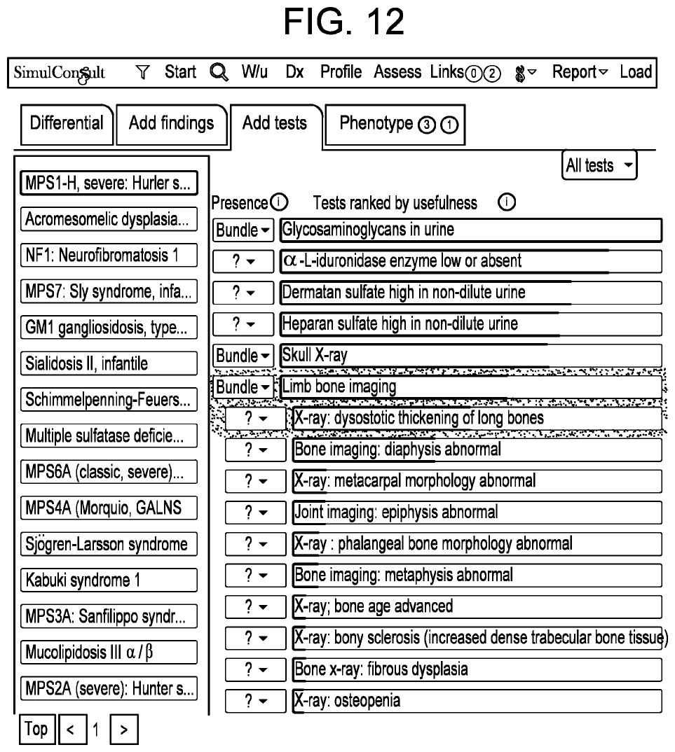

Accordingly, a primary care clinician could, in approximately one minute, send information to a specialist that is detailed, focused on useful findings, and computable. The specialist, after re-creating the case could display a list of useful clinical findings (“Add findings” tab) or lab findings (“Add tests” tab) from the full set of many thousands of findings in the software, as shown in FIG. 12. From the “Limb bone imaging” bundle the specialist could advise checking for dysostotic thickening of bone.

What is claimed is:

1. A method comprising the steps of:
(a) providing a physical computing device having stored therein a plurality of candidate medical conditions and lists of findings for a plurality of problem or medical specialty workups, wherein each of the findings is representative of clinical information about the medical conditions, and wherein the findings in the lists of findings are tagged with a metric of the usefulness of the finding in disambiguating among the plurality of medical conditions;
(b) specifying in the physical computing device a first finding as being present or absent in a patient in a first list of findings of a workup of the plurality of problem or medical specialty workups, wherein the physical computing device then automatically re-tags the findings in the first list of findings as a function of the usefulness of each finding in disambiguating among the plurality of medical conditions computed based on the specifying of the first finding;
(c) specifying in the physical computing device another finding either in the first list of findings or in a second list of findings of another problem or medical specialty workup as being present or absent in the patient, and recomputing the usefulness of unspecified findings in the first or second list;
(d) outputting a report of the findings specified in (b) and (c) indicating whether the finding is absent, or present organized by a computed rank of pertinence; and
(e) transmitting the report to a second user.

2. The method of claim 1, wherein the user is a primary care clinician.

3. The method of claim 2, wherein the second user is a specialist in the first medical specialty.

4. The method of claim 1, further comprising generating in the physical computing device a list of medical conditions ranked by likelihood.

5. The method of claim 1, wherein the report comprises a character string that allows re-creation of the list of medical conditions ranked by likelihood.

6. The method of any one of claims 1-5, wherein in step (c) the another finding is in the first list of findings.

7. The method of any one of claims 1-6, further comprising specifying in the physical computing device the age and/or sex of a subject.

8. The method of any one of claims 1-7, further comprising specifying an onset of the first or the another finding, when present.

9. The method of any one of claims 1-8, further comprising the step of ordering via the physical computing device a medical test to determine the presence or absence of an unspecified finding in the first or second list of findings.

10. The method of any one of claims 1-9, wherein the report comprises structured coding of the first and another findings to standardized coding lists.

11. The method of any one of claims 1-10, wherein the report is in the Subjective, Objective, Assessment, and Plan (SOAP) note format.

12. The method of any one of claims 1-11, further comprising the step of computing the pertinence of findings specified in (b) and (c) in distinguishing among the plurality of medical conditions.

13. The method of claim 9, further comprising the step of displaying a list of the medical tests tagged with a metric of usefulness in disambiguating among the plurality of medical conditions or a list of a subset of medical tests selected by the user.

14. The method of any one of claims 1-13, wherein the medical specialty is genetics, metabolism, mitochondrial, neurology, rheumatology, allergy, immunology, anesthesiology, dermatology, diagnostic radiology, emergency medicine, family medicine, internal medicine, nuclear medicine, obstetrics, gynecology, ophthalmology, pathology, pediatrics, physical medicine and rehabilitation, preventive medicine, psychiatry, radiation oncology, surgery, or urology.

15. The method of any one of claims 1-14, where the report comprises plain text that can be copied and pasted.

16. The method of any one of claims 1-15, wherein in step (c) the another finding is in the second list.

17. The method of claim 16, wherein the physical computing device highlights the name of the another workup to the user based on the usefulness of the findings in its list of findings to further characterize the patient.

18. A physical computing device having stored therein a plurality of candidate medical conditions and lists of findings for a plurality of problem or medical specialty workups, wherein each of the findings is representative of clinical information about the medical conditions and being programmed with executable instructions for (a) allowing a user to specify a first finding in a first list of findings for a workup of the plurality of problem or medical specialty workups as being present or absent in a patient; (b) calculating the usefulness of unspecified findings in the first list or a second list of findings of another problem or medical specialty workup based on the first finding; and (c) allowing the user to specify in the physical computing device another finding either in the first list of findings or in the second list of findings, and recomputing the usefulness of unspecified findings in the first or second list.

19. The device of claim 18, wherein in (a) the workup is a medical specialty workup and in (c) the another finding is in the second list.

20. The device of claim 18-19, further programmed to output a report of the findings from the first list indicating whether the finding is present, absent, or unknown, organized by pertinence rank.

21. The device of any one of claims 18-20, further programmed to compute the pertinence of findings specified in (a) and (c) in distinguishing among the plurality of medical conditions.

22. The device of any one of claims 18-21, further programmed to display a list of medical tests tagged with a metric of usefulness in disambiguating among the plurality of medical conditions or a list of medical tests selected by the user.

23. The device of any one of claims 18-22, further programmed to determine the usefulness of an unspecified finding in the second list and highlighting the name of the another workup to the user based on the usefulness of the findings in its list.

24. The device of any one of claims 18-23, further programmed to perform at least one of the following steps:
calculate and display a differential diagnosis;
create a report comprising computable information on a status of findings in the first and/or second lists;
create a report comprising a status and/or usefulness of the first finding;
create a report comprising a status and/or usefulness of all findings specified in (a) and (c);
create a report that is in SOAP note format;
create a report comprising computable information on a status of the findings specified in (a) and/or (c);
share a report with a second user; and/or
transfer computable information on a status of the findings specified in (a) and (c) from a report into the device.

25. A non-transient computer readable medium having stored therein a plurality of candidate medical conditions and lists of findings for a plurality of problem or medical specialty workups, wherein each of the findings is representative of clinical information about the medical conditions and executable instructions to direct a physical computing device to implement a method comprising (a) allowing a user to specify a first finding in a first list of findings for a workup of the plurality of problem or medical specialty workups as being present or absent in a patient; (b) calculating the usefulness of unspecified findings in the first list or a second list of findings of another problem or medical specialty workup based on the first finding; and (c) allowing the user to specify in the physical computing device another finding either in the first list of findings or in the second list of findings, and recomputing the usefulness of unspecified findings in the first or second list.

26. The medium of claim 25, wherein in (a) the workup is a medical specialty workup and in (c) the another finding is in the second list.

27. The medium of claim 25-26, wherein the instructions further direct the device to output a report of the findings in the first list indicating whether the finding is present, absent, or unknown, organized by pertinence rank.

28. The medium of any one of claims 25-27, wherein the instructions further direct the device to compute the pertinence of the findings specified in (a) and (c) in distinguishing among the plurality of medical conditions.

29. The medium of any one of claims 25-28, wherein the instructions further direct the device to display a list of medical tests tagged with a metric of usefulness in disambiguating among the plurality of medical conditions or a list of medical tests selected by the user.

30. The medium of any one of claims 25-29, wherein the instructions further direct the device to determine the usefulness of an unspecified finding in the second list and highlight the name of the another workup to the user based on the usefulness of the findings in its list.

31. The medium of any one of claims 25-30, wherein the instructions further direct the device to perform at least one of the following steps:
calculate and display a differential diagnosis;
create a report comprising computable information on a status of findings in the first and/or second lists;
create a report comprising a status and/or usefulness of all findings specified in (a) and (c);
create a report that is in SOAP note format;
create a report comprising computable information on a status of the findings specified in (a) and/or (c);
share a report with a second user; and/or
transfer computable information on a status of the findings specified in (a) and (c) from a report into the device.

32. A method of facilitating medical care comprising
(a) providing a physical computing device of any one of claims 18-24; and
(b) specifying in the physical computing device a first finding as being present or absent in a patient in a first list of findings for a workup;
(c) calculating the usefulness of unspecified findings in the first list or a second list of findings of another problem or medical specialty workup based on the first finding; and
(d) specifying in the physical computing device another finding either in the first list of findings or in the second list of findings, and recomputing the usefulness of unspecified findings in the first or second list.

33. The method of claim 32, wherein the physical computing device automatically re-tags the findings in the first list of findings as a function of the usefulness of each finding in disambiguating among the plurality of medical conditions computed based on the specifying of the first finding.

34. The method of claim 32-33, further comprising outputting a report of the findings from the first list indicating whether the finding is present, absent, or unknown, organized by pertinence rank.

35. The method of claim 34, further comprising transmitting the report to a second user.

36. The method of any one of claims 32-35, further comprising generating in the physical computing device a list of medical conditions ranked by likelihood.

37. The method of claim 34, wherein the report comprises a character string that allows re-creation of the list of medical conditions ranked by likelihood.

38. The method of any one of claims 32-37, wherein in step (d) the another finding is in the first list of findings.

39. The method of any one of claims 32-38, further comprising specifying in the physical computing device the age and/or sex of a subject.

40. The method of any one of claims 32-39, further comprising specifying an onset of the first or another finding, when present.

41. The method of any one of claims 32-40, further comprising the step of ordering via the physical computing device a medical test to determine the presence or absence of an unspecified finding in the first or second list of findings.

42. The method of claim 34, wherein the report comprises structured coding of the findings specified in (a) and (d) to standardized coding lists.

43. The method of claim 34, wherein the report is in the Subjective, Objective, Assessment, and Plan (SOAP) note format.

44. The method of any one of claims 32-43, further comprising the step of computing the pertinence of the findings specified in (a) and (c) in distinguishing among the plurality of medical conditions.

45. The method of claim 32, further comprising the step of displaying a list of the medical tests tagged with a metric of usefulness in disambiguating among the plurality of medical conditions or a list of medical tests selected by the user.

46. The method of claim 34, where report comprises a character string that allows re-creation of the list of medical conditions ranked by likelihood.

47. The method of claim 34, where the report comprises plain text that can be copied and pasted.

48. The method of any one of claims 32-47, wherein the another finding is in the second list.

49. The method of claim 48, wherein the physical computing device highlights the name of the another workup to the user based on the usefulness of the findings in its list of findings to further characterize the patient.
